(19) United States
(12) Patent Application Publication
(10) Pub. No.: US 2007/0270815 A1
(43) Pub. Date: Nov. 22, 2007

(54) Bone anchors with end-loading receivers for elongated connecting elements in spinal surgical procedures

(76) Inventors: Chris Johnson, Germantown, TN (US); Hugues Malandain, Mountain View, CA (US)

Correspondence Address: KRIEG DEVAULT LLP, ONE INDIANA SQUARE, SUITE 2800, INDIANAPOLIS, IN, 46204-2709, US

(21) Appl. No.: 11/408,219

(22) Filed: Apr. 20, 2006

(51) Int. Cl. A61F 2/30

(52) U.S. Cl. 606/61

(57) ABSTRACT

Devices and methods include an anchor assembly engageable to a vertebra and a connecting element positionable through a receiver of the anchor assembly. The receiver includes a body extending along a longitudinal axis that defines an axial bore extending longitudinally therein and opening at least distally and a trans-axial bore that opens in opposite sides of the receiver. The connecting element can be positioned through the trans-axial bore in an endwise manner through the receiver. The side openings of the trans-axial bore are encompassed by the body of the receiver on all sides of each opening.

### BACKGROUND

Elongated connecting elements, such as rods, plates, tethers, wires, cables, and other devices have been implanted along the spinal column and connected between two or more anchors engaged between one or more spinal motion segments. Such connecting elements can be positioned in the anchor with a top-down approach or a side-to-side approach. In the top-down approach, the incision extends between the anchors, and the connecting element is moved distally or toward the opposite side of the patient through the incision until it is positioned for engagement to the anchors. For example, in posterior spinal surgery, the connecting element is moved anteriorly through a posterior incision to the anchors. In the side-to-side approach, the connecting element is positioned distally through the incision to a location adjacent the anchors, and then moved in a sideways direction until it is positioned for engagement with the anchors. For example, in posterior spinal surgery, the connecting element can be moved anteriorly through a posterior incision to a location adjacent the anchor, and then moved medially or laterally, depending on the relative anchor location, into an opening into the receiver. To prevent the connecting element from passing back out of the opening and to secure the connecting element to the receiver, a set screw or nut can be engaged to the receiver to block the opening.

Other surgical instruments and techniques contemplate insertion of a connecting element to an anchor engaging position along a path that extends along or is generally parallel an anchor alignment axis extending between the anchors. Examples of such instruments and techniques are provided in U.S. Pat. No. 6,530,929, which is incorporated herein by reference in its entirety.

### SUMMARY

The present invention generally relates to devices and methods for securing connecting elements with bone anchor assemblies.

In one form, a spinal stabilization system includes an anchor member with a distal bone engaging portion to engage a vertebral body and a receiver at a proximal end of the anchor member extending along a longitudinal axis between a proximal end and a distal end. The receiver includes a distal ring portion and a pair of arms extending proximally from the distal ring portion to a proximal ring portion extending about proximal ends of the pair of arms. The proximal ring portion extends completely about and defines a proximal portion of an axial bore extending therethrough. The axial bore extends along the longitudinal axis along the pair of arms and through a distal opening of the distal ring portion. The receiver further defines a trans-axial bore extending transversely to the axial bore. The trans-axial bore opens at opposite sides of the pair of arms with the openings each circumscribed by the pair of arms, the proximal ring portion and the distal ring portion. The proximal and distal ring portions and the pair of arms define a unitary body structure for the receiver. The proximal head of the anchor member is pivotally captured in the distal ring portion with the distal engaging portion extending distally and axially through the distal opening of the distal ring portion. The elongated connecting element extends through the trans-axial bore.

In another form, a spinal stabilization system includes an anchor member with a distal bone engaging portion to engage a vertebral body and a receiver at a proximal end of the anchor member that extends along a longitudinal axis between a proximal end and a distal end of a body of the receiver. The receiver includes an unitary, integral body that defines an axial bore extending along the longitudinal axis that opens at the distal and proximal ends that is completely encircled at the distal and proximal ends with the body. The body further defines a trans-axial bore extending therethrough that intersects the axial bore and opens at opposite sides of the body. The side openings of the trans-axial bore are completely encircled by the body and the proximal head of the anchor member is pivotally captured in the body with the distal engaging portion extending distally and axially through the distal opening of the body. An elongated connecting element extends through the trans-axial bore.

In another aspect, a method for stabilizing a spinal column segment comprises: engaging an anchor assembly to a vertebra of the spinal column, the anchor assembly comprising a receiver extending along a longitudinal axis between a distal end and a proximal end and an anchor member with a distal bone engaging portion extending distally and axially from the distal end of the receiver; pivoting the receiver to orient the longitudinal axis in a desired position relative to the distal bone engaging portion; inserting a connecting element through a trans-axial bore of the receiver so that the connecting element extends from the receiver in at least one direction therefrom that is transverse to the longitudinal axis; and retaining the connecting element distally and proximally within the receiver with a unitary body structure of the receiver that includes a proximal ring portion extending about the proximal end of the receiver and a distal ring portion extending about the distal end of the receiver and a pair of arms extending between the proximal and distal ring portions on opposite sides of the connecting element.

These and other aspects will be discussed further below.

### BRIEF DESCRIPTION OF THE DRAWINGS

FIG. 1 is a posterior elevation view of a spinal column segment and spinal implant system.

FIG. 2 is an elevation view of an anchor assembly.

FIG. 3 is an exploded view of the anchor assembly of FIG. 2.

FIG. 4 is a perspective view in partial section showing a proximal portion of the anchor assembly of FIG. 2.

FIG. 5 is an elevation view of a receiver of the anchor assembly of FIG. 2.

FIG. 6 is a side view of the receiver of FIG. 5.

FIG. 7 is a section view along line 7-7 of FIG. 6.

FIG. 8 is a perspective view of an engaging member engageable with the receiver to secure a connecting element in the receiver.

FIG. 9 is a diagrammatic elevation view of a spinal column segment showing one embodiment insertion instrument mounted to anchor assemblies engaged to adjacent vertebrae and a connecting element before insertion through the anchor assemblies.

FIG. 10 is the view of FIG. 9 with the connecting element inserted through the anchor assemblies.

### DESCRIPTION OF THE ILLUSTRATED EMBODIMENTS

For the purposes of promoting an understanding of the principles of the invention, reference will now be made to the embodiments illustrated in the drawings and specific language will be used to describe the same. It will nevertheless be understood that no limitation of the scope of the invention is thereby intended. Any such alterations and further modifications in the illustrated devices, and such further applications of the principles of the invention as illustrated herein are contemplated as would normally occur to one skilled in the art to which the invention relates.

Devices and methods for facilitating placement and securement of a connecting element between anchor assemblies are provided for spinal surgical procedures. The anchor assemblies each include a receiver having a first bore extending along a first axis and a second bore extending along a second axis that intersects the first axis. The first and second bores are each completely encircled or surrounded by the receiver to capture the connecting element in the trans-axial bore. In one form, an engaging member is engageable to the receiver in the axial bore to further secure the connecting element with the anchor assembly. The interconnection of the portions of the receiver about all sides of the axial and trans-axial bores can reduce the profile of the receiver and prevent splaying of portions of the receiver in response to insertion of the connecting element and/or engagement of the engaging member to the receiver. Since the trans-axial bore opens only on the opposite sides of the receiver, the elongate connecting element can be inserted through the trans-axial bore in an endwise fashion along an insertion axis that corresponds generally to the axis of the trans-axial bore. The connecting element can be moved in the patient along the insertion axis for engagement with one or more receivers of one or more other anchor assemblies.

The anchor assemblies discussed herein can be multi-axial or uni-axial in form, and can include an anchor member engageable to a vertebra and a receiver for receiving a connecting element. The multi-axial anchor assemblies allow the anchor member coupled to the receiver to be positioned at various angles relative to the receiver. The uni-axial anchor assemblies can also provide a fixed positioning of the receiver relative to the anchor member. The anchor member of the anchor assemblies forms a distal lower portion that is engageable to a vertebral body with the proximal receiver positioned adjacent the vertebra. The anchor member can extend along the longitudinal axis of the receiver to minimize the footprint of the anchor assembly and minimize intrusion into adjacent tissue. In one embodiment, the anchor member is in the form of a bone screw with a threaded shaft and a proximal head that is pivotally captured in the receiver. In other embodiments, the distal anchor member can be in the form of a hook, staple, cable, tether, suture anchor, interbody fusion implant, artificial disc implant, bolt, or other structure engageable to bony tissue. The receiver defines a passage that receives a connecting element, such as a rod, tether, wire, cable, plate or other elongated connecting element that can extend between one or more additional anchor assemblies secured to one or more additional vertebrae.

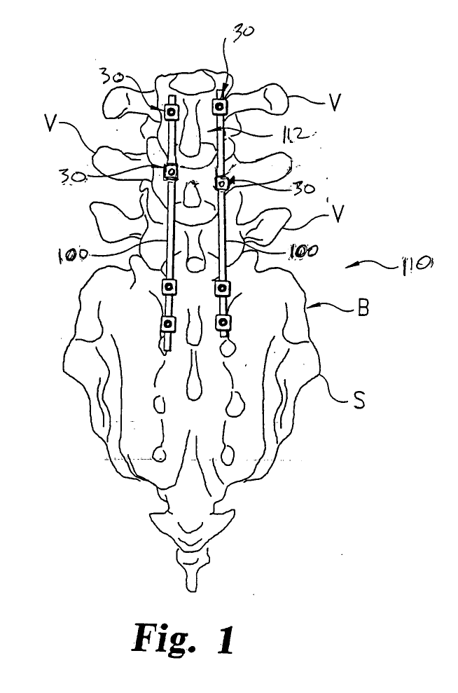

FIG. 1 illustrates a posterior spinal implant system 110 located along a spinal column of a patient. More specifically, implant system 110 can be affixed to bones B of the spinal column segment 112 from a posterior approach. Bones B can include the sacrum S and several vertebrae V. Implant system 110 generally includes several bone anchor assemblies 30 and elongated connecting elements 100 structured to selectively interconnect with bone anchor assemblies 30. Connecting elements 100 may be a spinal rod, plate, bar, or other elongated element having a length to extend between at least two vertebrae. Connecting element 100 may be solid or hollow along some or all of its length and/or may be of homogenous or heterogeneous composition. In implant system 110, bone anchor assemblies 30 are affixed to various locations of the spinal column segment 112 and interconnected with connecting elements 100. Spinal implant system 110 may be used for, but is not limited to, treatment of degenerative spondylolisthesis, fracture, dislocation, scoliosis, kyphosis, spinal tumor, and/or a failed previous fusion.

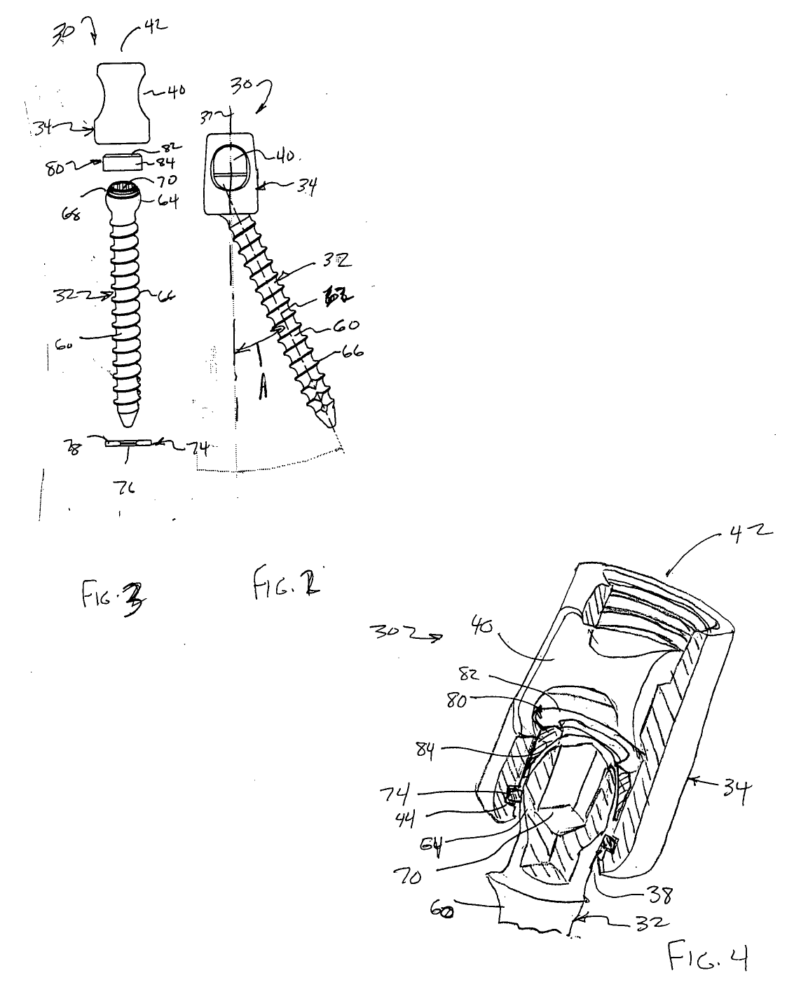

FIGS. 2-4 show anchor assembly 30 in further detail. Anchor assembly 30 includes an anchor member 32 pivotally coupled to receiver 34 with receiver 34 adjacent a proximal end of anchor member 32 and anchor member 32 extending distally from receiver 34. Anchor member 32 includes an elongated shaft 60 extending along a longitudinal axis 62 and an enlarged, spherical head 64 at the proximal end of shaft 60. Shaft 60 can include an outer thread profile 66 for threadingly engaging a bony structure to secure anchor assembly 30 thereto. Enlarged head 64 can include a proximal end face with teeth 68 extending circumferentially thereabout and a tool recess 70 extending distally therein along axis 62. Tool recess 70 can receiver a driving tool to facilitate engagement of shaft 60 to the bony structure.

The threaded shaft 60 can be provided with self-drilling and/or self-tapping thread profile to facilitate insertion into bony tissue. In another embodiment, the threaded shaft is configured for insertion in a pre-drilled and pre-tapped hole in the vertebral body. Shaft 62 can be sized and shaped to engage a pedicle of a vertebral body, although other sizes and shapes are also contemplated.

A retaining ring 74 can be positioned adjacent a proximal end of shaft 60 and engaged with receiver 34 in an inner groove 44 thereof that is adjacent to and about a lower or distal opening 38. Retaining ring 74 can include a ring-shaped body 78 and a central bore 76 for receiving shaft 60 therethrough distally from receiver 34. Ring-shaped body 78 can be sized relative to head 64 to support head 64 when in contact with a distally oriented surface of head 64. Other embodiment contemplate head 64 is supported by a lip or flange that is formed integrally with receiver 34 about opening 38.

Anchor assembly 30 can also include a crown 80 positioned about the proximal side of head 64 in receiver 34 when head 64 is captured in receiver 34. Crown 80 includes a proximal end wall 82 and a circumferential and distally extending sidewall 84 that extends about head 64. Crown 80 can also include a bore through proximal end wall 82 to receive a driving instrument that is engaged in recess 70 of head 64. Sidewall 84 forms a cavity or recess to receive head 64 while allowing head 64 and thus shaft 60 to pivot in receiver 34. Crown 80 can be firmly seated against head 64 when the connecting element is engaged against the proximal end wall 82. The teeth 68 of head 64, if provided, can bite into crown 80 at an inner surface of proximal end wall 82 to lock anchor member 32 in position relative to crown 80. In another embodiment, head 64 can be smooth and engaged with crown 80 so that pivoting of anchor member 32 is possible even when connecting element 100 is engaged against crown 80.

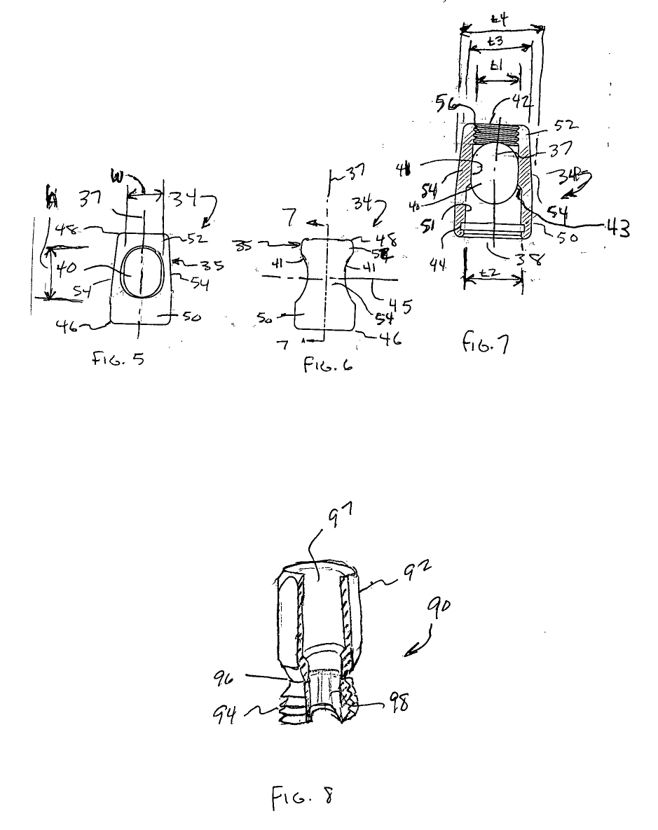

Receiver 34 is also shown in isolation in FIGS. 5-7. Receiver 34 includes a tubular body 35 that extends along a longitudinal axis 37 between a distal end 46 and a proximal end 48. Tubular body 35 extends about first axial bore 42, and axial bore 42 can open at proximal end 48 and define opening 38 at distal end 46. Tubular body 35 further defines trans-axial bore 40 that extends along a connecting element insertion axis 45 between opposite sides of body 35. Connecting element 100 can be positioned through trans-axial bore 40 in a direction that generally parallels insertion axis 45. Connecting element 100 is inserted in an endwise fashion through one of the side openings of trans-axial bore 40 since bores 40, 42 are encircled by body 35, preventing side-loading of connecting element 100 therein.

Anchor member 32 can be pivotally captured in receiver 34 so that receiver 34 can be pivoted to anchor member 32. As shown in FIG. 2, shaft 60 can be pivoted from a location along longitudinal axis 37 to an angle A or to any pivoted location therebetween. Accordingly, there is provided an infinite number of angular orientations between anchor member axis 62 and longitudinal axis 37 defined by a cone having an apex at head 64. Other embodiments contemplated that the anchor member 32 is pivotal in a single plane, or in selected planes, relative to receiver 34. When anchor member 32 is engaged to the bony tissue, receiver 34 can be pivotally adjusted and repositioned as needed for engagement with connecting element 100 and to accommodate engagement with of anchor assembly 30 with the vertebral anatomy. In another embodiment, receiver 34 is integral with and formed as a single piece with the anchor member 32, providing a uni-axial anchor assembly 30. Furthermore, a set screw, washer, crown, cap or other engaging member may be provided for engagement within and/or about receiver 34 to secure connecting element 100 thereto.

Receiver 34 includes a low profile outer surface since bores 40, 42 are encircled by respective portions of body 35, maximizing the strength of receiver 34 while minimizing the amount of material comprising it. Body 35 includes a unitary, integral structure that includes a lower or distal ring portion 50 and an upper or proximal ring portion 52 interconnected by arm portions 54. Arm portions 54 are integrally formed with distal ring portion 50 and proximal ring portion 52 at opposite ends thereof and extend along opposite sides of trans-axial bore 40. Distal ring portion 50, proximal ring portion 52 and arms 54 define the opposite side openings 41 of trans-axial bore 40, while proximal ring portion 52 defines the proximal end opening of axial bore 42. The inner surface of proximal ring portion 52 includes a thread profile 56 extending along longitudinal axis 37 about axial bore 42 to threadingly receive an engaging member, such as engaging member 90 discussed below. Distal ring portion 50 defines distal opening 38, and includes an inner surface 51 defining groove 44 extending about axial bore 42.

As shown in FIG. 7, providing bores 40 and 42 with an encircled configuration allows material removal from body 35 to provide a low profile arrangement for receiver 34. In particular, axial bore 42 can be formed with an internal transverse dimension t1 transversely to longitudinal axis 37 along proximal ring portion 52 and arms 54, and a second, greater transverse dimension t2 transversely to longitudinal axis 37 along distal ring portion 50. Transverse dimension t2 can accommodate the head of anchor member 32 while transverse dimension t1 is provided to accommodate the width of the connecting element 100 when positioned through trans-axial bore 40. Since arm portions 54 are connected at their proximal ends with proximal ring portion 52, material thickness of the arm portions 54 at distal ring portion 50 can be reduced since bending stresses at the junction therewith can be distributed to the proximal ring portion 52.

Furthermore, proximal ring portion 52 includes an outer transverse width t3 that is less than an outer transverse width t4 defined by distal ring portion 50. Arm portions 54 include a width that provides a smooth taper from width t4 to width t3 and form body 35 with a smooth outer surface for body 35 that contacts adjacent tissue to minimize irritation. The smooth surface profile and relative dimensions of the external surfaces can be carried out about the entire perimeter of body 35 except where interrupted by side openings 41, as shown in FIG. 5. The transition of the inner surfaces of bore 42 between arm portions 54 and distal ring portion 50 can be abrupt, as shown in FIG. 7 at lip 45, since these internal surfaces are not in contact with tissue.

As shown in FIG. 5, trans-axial bore 40 can be provided with end openings 41 between arm portions 54 that have a height h along longitudinal axis 37 and width w transverse to longitudinal axis 37. Height h can be greater than width w to allow variable placement of the connecting element 100 in trans-axial bore 40 with respect to its location along axis 37. The engagement member can force the connecting element 100 distally along longitudinal axis 37 and into contact with crown 80 when secured to the threads in proximal ring portion 52. Alternatively, the connecting element can simply be retained in receiver 34 without an engaging member, and is captured in trans-axial bore 40 by distal ring portion 50, proximal ring portion 52 and arm portions 54 while being allowed to float along longitudinal axis 37 in response to motion of the spinal column segment to which it is attached.

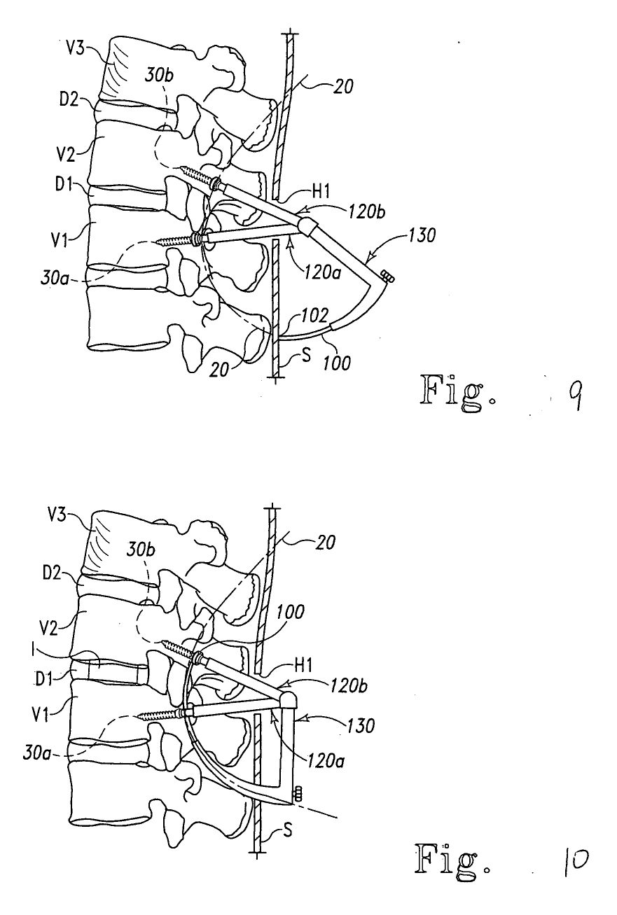

FIG. 8 shows one embodiment engaging member 90 engageable with receiver 34 to secure connecting element 100 therein. Engaging member 90 includes a proximal head portion 92 and a distal engaging portion 94. Head portion 92 can be coupled with engaging portion 94 at a reduced thickness break-off region 96. Head portion 92 can further include a receptacle 97 extending axially therealong. Receptacle 97 is in communication with a second receptacle 98 extending axially into engaging portion 94.

Engaging portion 94 can include external threads that threadingly engage threads along axial bore 42 of receiver 34. Engaging portion 94 is positionable into contact with the connecting element 100 in trans-axial bore 40 to force the connecting element 100 against crown 80, thereby seating and substantially fixing bone anchor 32 in position receiver 34. When a threshold torque applied to head portion 92, it severs from engaging portion 94 at break-off region 96. Should further tightening of engaging portion 94 be desired, or should it be desired to remove or loosen engaging portion 94, a driving instrument can be positioned into second receptacle 98 to deliver the required rotational forces.

Referring now to FIGS. 9-10, one embodiment insertion technique for positioning a connecting element 100 through a pair of anchor assemblies 30a, 30b will be discussed. The spinal column segment is shown with three vertebrae V1, V2, V3. Disc space D1 is located between vertebrae V1 and V2, and disc space D2 is located between vertebrae V2 and V3. A first anchor assembly 30a is engaged to vertebra V1 and a second anchor assembly 30b is engaged to vertebra V2. A first extender 120a is coupled to first anchor assembly 30a, and a second extender 120b is coupled to second anchor assembly 30b. Extenders 120a, 120b can be manipulated so that their proximal ends are adjacent one another for mounting of an inserter instrument 130.

For minimally invasive procedures, anchor assemblies 30a, 30b can be engaged to the respective vertebrae V1, V2 through percutaneous pathways formed through skin S and/or the tissue between skin S and the vertebrae. A separate pathway can be provided for each anchor or, as illustrated, a single pathway H1 is provided for both anchors. Connecting element 100 is mounted to inserter instrument 130, and can be guided along insertion axis 20 from a location outside the patient's body, as shown in FIG. 9, to a location extending between anchor assemblies 30a, 30b as shown in FIG. 10.

The receivers 34 of anchor assemblies 30a, 30b are oriented so that trans-axial bores 40 face one another and are aligned in the direction from which connecting element 100 will be received for positioning through the trans-axial bores 40. The elongated arrangement of the trans-axial bores 40 in the proximal and distal directions provides space for guiding the insertion of connecting element 100 into the receivers 34 of the anchor assemblies and space for reduction of the connecting element into the receiver after positioning in the receiver.

In the illustrated embodiment, connecting element 100 has a leading insertion end 102 that can be tapered and connecting element 100 is moved in the caudal to cephalad direction for insertion. It is contemplated that the connecting element can be inserted in the cephalad to caudal direction, in the medial-lateral directions for transverse connectors, or in any other direction suitable for placement of a connecting element in a spinal procedure. Furthermore, the connecting element can be engaged to more than two anchor assemblies along the spinal column, and provide stabilization for multiple spinal motion segments. The connecting element can also be positioned for engagement with a single anchor assembly engaged to the spinal column, or for procedures involving single vertebra.

Connecting element 100 can be a spinal rod connectable to one or more anchor assemblies to rigidly stabilize the spinal column. Connecting element 100 can also be flexible to allow motion of the spinal motion segment or segments to which it is attached. Other embodiments contemplate that connecting element 100 can comprise multiple components. In another embodiment, the connecting element includes a carrier for a non-rigid implant such as a tether, and the carrier is employed to facilitate placement of the non-rigid connecting element through the anchor assembly in endwise fashion. Other embodiments contemplate other forms for the connecting element, including plates, wires, struts, cables, and other devices capable of endwise insertion through the receiver of the anchor assembly, either alone or via a carrier.

In one procedure, anchor assembly 30 can be inserted through a minimally invasive access portal for engagement of anchor member 32 with bony tissue of a vertebra, such as the pedicle of the vertebra. The minimally invasive access portal can be provided by a micro-incision, a sleeve, a sleeve with an expandable working channel, a retractor blade, or two or more retractor blades of a retractor system. The low profile of receiver 34 and axial alignment of anchor member 32 therewith can enhance the minimally invasive character of the procedures. Anchor assembly 30 can be guided to position anchor member 32 in a desired trajectory or path into the vertebra using fluoroscopic imaging, endoscopic viewing, or other suitable viewing or imaging systems. Anchor member 32 can be engaged to the vertebra by positioning a driving instrument through axial bore 42 and into engagement with anchor member 32 in retaining portion 36. The procedure can be repeated for one or more additional anchor assemblies engaged to the same vertebrae, or to different vertebrae, along one or more spinal motion segments.

In one procedure, extenders can be mounted to anchor assemblies 30 either before or after engagement of anchor member 32 to the adjacent vertebrae. The extenders extend from the anchor member to a proximal end located outside the patient. An insertion instrument for delivering the connecting element along the insertion axis to the anchor assemblies is provided that can be mounted to the proximal end of one or more of the extenders. The inserter is operable to pivot about the proximal ends of the extenders to deliver the connecting element along the insertion axis and through the passage of one or more of the receivers of the anchor assemblies.

Other embodiments for procedures contemplate other insertion techniques for the connecting element, including free hand insertion of the connecting element along the insertion axis, insertion instruments that are not coupled to the anchor assemblies; insertion instruments employing image-guided navigation system; insertion instruments that deliver the connecting element through tissue without retraction or cutting of the tissue; and insertion instruments that deliver the connecting element through open incisions where skin and tissue are retracted to accommodate placement of the connecting element along the insertion axis, for example.

While the invention has been illustrated and described in detail in the drawings and foregoing description, the same is to be considered as illustrative and not restrictive in character, it being understood that all changes and modifications that come within the spirit of the invention are desired to be protected.

What is claimed is:

1. A spinal stabilization system, comprising:
an anchor member engageable to a vertebral body, said anchor member including a distal bone engaging portion and a head at a proximal end of said distal bone engaging portion;
a receiver at a proximal end of said anchor member extending along a longitudinal axis between a proximal end and a distal end, said receiver including a distal ring portion, a pair of arms extending proximally from said distal ring portion to a proximal ring portion extending about proximal ends of said pair of arms, said proximal ring portion extending completely about and defining a proximal portion of an axial bore extending therethrough, said axial bore extending along said longitudinal axis along said pair of arms and through a distal opening of said distal ring portion, said receiver further defining a trans-axial bore extending transversely to said axial bore, said trans-axial bore opening at opposite sides of said pair of arms with said openings each circumscribed by said pair of arms, said proximal ring portion and said distal ring portion, wherein said proximal and distal ring portions and said pair of arms define a unitary body structure for said receiver, wherein said proximal head of said anchor member is pivotally captured in said distal ring portion with said distal engaging portion extending distally and axially through said distal opening of said distal ring portion; and
an elongated connecting element extending through said trans-axial bore.

2. The system of claim 1, wherein said distal bone engaging portion is an elongated, externally threaded shaft.

3. The system of claim 2, further comprising a crown in said receiver between said proximal head and said elongated connecting element.

4. The system of claim 1, wherein said proximal ring portion includes a thread profile extending about an inner surface thereof and about said axial bore, and further comprising an engaging member with an externally threaded body positionable in threaded engagement with said thread profile to secure said connecting element in said trans-axial bore.

5. The system of claim 1, wherein said proximal ring portion defines a first external width transversely to said longitudinal axis and said distal ring portion defines a second external width transversely to said longitudinal axis, and said arm portions define an external width tapering from said second external width of said distal ring portion to said first external width of said proximal ring portion.

6. The system of claim 5, wherein said axial bore defines a first internal width transversely to said longitudinal axis along said proximal ring portion of said axial bore and said axial bore defines a second internal width transversely to said longitudinal axis along said distal ring portion that is greater than said first width.

7. The system of claim 6, wherein said second width extends through said distal opening.

8. The system of claim 7, wherein said distal ring portion includes an inner surface extending about said longitudinal axis defining said second width, said inner surface defining a groove about said distal opening and further comprising a retaining member in said groove supporting said proximal head in said distal ring portion.

9. The system of claim 1, wherein said openings of said trans-axial bore each include a first dimension along said longitudinal axis and a second dimension orthogonally to said longitudinal axis, wherein said first dimension is greater than said second dimension.

10. The system of claim 1, wherein said receiver and said anchor member are pivotally coupled to one another.

11. A spinal stabilization system, comprising:
an anchor member engageable to a vertebral body, said anchor member including a distal bone engaging portion and a proximal head at a proximal end of said distal bone engaging portion;
a receiver at a proximal end of said anchor member including a body extending along a longitudinal axis between a proximal end and a distal end, wherein said body includes a unitary, integral structure and defines an axial bore extending along said longitudinal axis opening at said distal and proximal ends that is completely encircled at said distal and proximal ends with said body, said body further defining a trans-axial bore extending therethrough intersecting said axial bore and opening at opposite sides of said body, said side openings of said trans-axial bore being completely encircled by said body, wherein said proximal head of said anchor member is pivotally captured in said body with said distal engaging portion extending distally and axially through said distal opening of said body; and
an elongated connecting element extending through said trans-axial bore.

12. The system of claim 11, wherein said body of said receiver includes a distal ring portion extending about said axial bore, a pair of arms extending proximally from said distal ring portion to a proximal ring portion extending about said axial bore at proximal ends of said pair of arms, said pair of arms extending along opposite sides of each of said side openings of said trans-axial bore.

13. The system of claim 12, wherein said proximal ring portion includes a thread profile extending about an inner surface thereof and about said axial bore, and further comprising an engaging member with an externally threaded body positionable in threaded engagement with said thread profile to secure said connecting element in said trans-axial bore.

14. The system of claim 12, wherein said proximal ring portion defines a first external width transversely to said longitudinal axis and said distal ring portion defines a second external width transversely to said longitudinal axis, and said arm portions define an external width tapering from said distal ring portion to said proximal ring portion.

15. The system of claim 11, wherein said axial bore defines a first internal width transversely to said longitudinal axis along a proximal portion of said axial bore and said axial bore defines a second width transversely to said longitudinal axis at a proximal end of said axial bore, wherein said second width is greater than said first width.

16. The system of claim 15, wherein said second width extends through said distal opening.

17. The system of claim 11, wherein said side openings of said trans-axial bore each include a first dimension along said longitudinal axis and a second dimension orthogonally to said longitudinal axis, wherein said first dimension is greater than said second dimension.

18. A method for stabilizing a spinal column segment, comprising:
engaging an anchor assembly to a vertebra of the spinal column, the anchor assembly comprising a receiver extending along a longitudinal axis between a distal end and a proximal end and an anchor member with a distal bone engaging portion extending distally and axially from the distal end of the receiver;
pivoting the receiver to orient the longitudinal axis in a desired position relative to the distal bone engaging portion;
inserting a connecting element through a trans-axial bore of the receiver so that the connecting element extends from the receiver in at least one direction therefrom that is transverse to the longitudinal axis; and
retaining the connecting element distally and proximally within the receiver with a unitary body structure of the receiver that includes a proximal ring portion extending about the proximal end of the receiver and a distal ring portion extending about the distal end of the receiver and a pair of arms extending between the proximal and distal ring portions on opposite sides of the connecting element.

19. The method of claim 18, wherein the trans-axial bore is elongated in the distal and proximal directions and further comprising allowing the connecting element to move in the trans-axial bore in the distal and proximal directions in response to movement of the spinal column segment.

20. The method of claim 18, further comprising:
securing the elongated connecting element in the trans-axial bore of the receiver with an engaging member engaged in a longitudinally extending and proximally opening axial bore of the receiver that extends through the proximal ring portion and is in communication with the trans-axial bore.

21. The method of claim 20, wherein securing the connecting element engages the connecting element against a crown in the receiver, the crown being positioned between the connecting element and a head of the anchor member in the receiver.

22. The method of claim 18, wherein inserting the connecting element includes guiding a leading end of the connecting element through the trans-axial bore from a location remote from the receiver.
